(19) United States
(12) Patent Application Publication
(10) Pub. No.: US 2006/0278255 A1
(43) Pub. Date: Dec. 14, 2006

(54) Method and unit for cleaning pieces contaminated with organic matter

(76) Inventors: Henri Drogue, Trefflean (FR); Martin Goibier, Sene (FR); Salvatore Augeri, Vannes (FR); Thierry Garcia, Ramboullet (FR)

Correspondence Address: YOUNG & THOMPSON, 745 SOUTH 23RD STREET, 2ND FLOOR, ARLINGTON, VA, 22202, US

(21) Appl. No.: 10/556,824

(22) PCT Filed: May 12, 2004
(86) PCT No.: PCT/FR04/01155

§ 371 Date: May 15, 2006

(30) Foreign Application Priority Data
May 16, 2003 (FR) 03/05864

(51) Int. Cl. B08B 7/04; B08B 9/00; B08B 9/027; C23G 5/00; B08B 3/00

(52) U.S. Cl. 134/22.1; 134/22.11; 134/40; 134/166 C; 134/166 R; 134/10; 134/111

(57) ABSTRACT

In a method and a unit for cleaning pieces contaminated with organic matter using a cleaning fluid, at least a part of the cleaning fluid circulates in a loop between a unit (1) for cleaning pieces, in which the cleaning fluid is charged with organic material on contacting the pieces and a processing unit in which living microorganisms biologically decompose the organic matter contained in the fluid leaving the cleaning unit (1). The method includes an at least partial sterilisation of at least a part of the cleaning fluid circulating in the installation, for limiting or preventing the presence of living microorganisms in the cleaning fluid serving the cleaning unit (1). The method and apparatus apply to the degreasing of pieces.

The present invention relates to a process and an installation for the cleaning, in particular degreasing or de-oiling, of pieces soiled with organic material and bio-pollution.

The treatment by bacteria of fluids in the field of the treatment of wastewater is well known as shown, by way of prior art, in U.S. Pat. No. 5,245,246.

For numerous years there have been systems to clean objects contaminated with organic residues. For a long time there were used as cleaning fluids halogenated solvents or solvents constituted by mineral oil which require taking a large number of precautions for their handling or their elimination, these operations being costly.

To limit the risks connected with the use of such solvents, of which the most frequent were constituted by risk of burning, pollution, dermatosis and respiratory maladies, it was proposed to clean such pieces by means of a cleaning fluid adapted on the one hand to become loaded with organic material in contact with the pieces and on the other hand to be purified by means of microorganisms adapted to feed on the contaminating organic materials contained in the cleaning fluid. Such installations were particularly described in the patents DE 4209052 and EP 0 784 518. If such a solution solved the drawbacks mentioned above during the use of halogenated or non-halogenated solvents, it gave rise to other risks, in particular because of the presence of microorganisms. Thus the possibility cannot be excluded, that an operator having to handle a flow of cleaning fluid and the pieces to be cleaned, would be placed in contact with the microorganisms contained in the cleaning fluid. However, the presence in this fluid of pathogenic microorganisms supplied by the pieces or the dirt, could not be totally excluded. These foreign microorganisms have the danger of giving the operator maladies of the type of infection or allergy which can be more or less serious.

An object of the present invention is thus to provide a process and an installation of the type mentioned, whose characteristics permit avoiding or reducing, particularly in the course of the cleaning operation of the pieces, the risks of contamination of an operator connected to the contact of the operator with microorganisms adapted to purify the cleaning fluid or with other microorganisms supplied by the pieces or by the contamination.

To this end, the invention has for its object a process for the cleaning, in particular the degreasing or de-oiling, of pieces soiled with organic material, by means of a cleaning fluid of which at least a portion circulates in a loop between a washing unit for the pieces in which the cleaning fluid becomes loaded with organic materials in contact with the pieces, and a processing unit in which living microorganisms degrade biologically the organic material contained in the fluid from the washing unit, which process is characterized in that it consists in subjecting at least a portion of the cleaning fluid circulating in said installation to at least a partial sterilization so as to limit or even to avoid, the presence of living microorganisms in the cleaning fluid serving in the washing unit.

The invention also has for an object an installation for the cleaning, in particular the degreasing or de-oiling, of pieces soiled with organic material, by means of a cleaning fluid of which at least a portion circulates in a loop between a washing unit for the pieces in which the cleaning fluid becomes loaded with organic materials in contact with the pieces, and a processing unit in which living microorganisms degrade biologically the organic material contained in the fluid from the washing unit, characterized in that the installation moreover comprises, in the circuit for the circulation of fluid, means for at least partially sterilizing the microorganisms contained in at least a portion of the cleaning fluid.

Thanks to the partial or total sterilization of the cleaning fluid circulating within the installation, it is possible to limit or even avoid any contact of the hands of an operator with living microorganisms during the cleaning operation of the pieces, so as to avoid any risk of infection or allergies connected with contact with the microorganisms present in the cleaning fluid.

The invention will be better understood from a reading of the following description of embodiments, with reference to the accompanying drawings, in which:

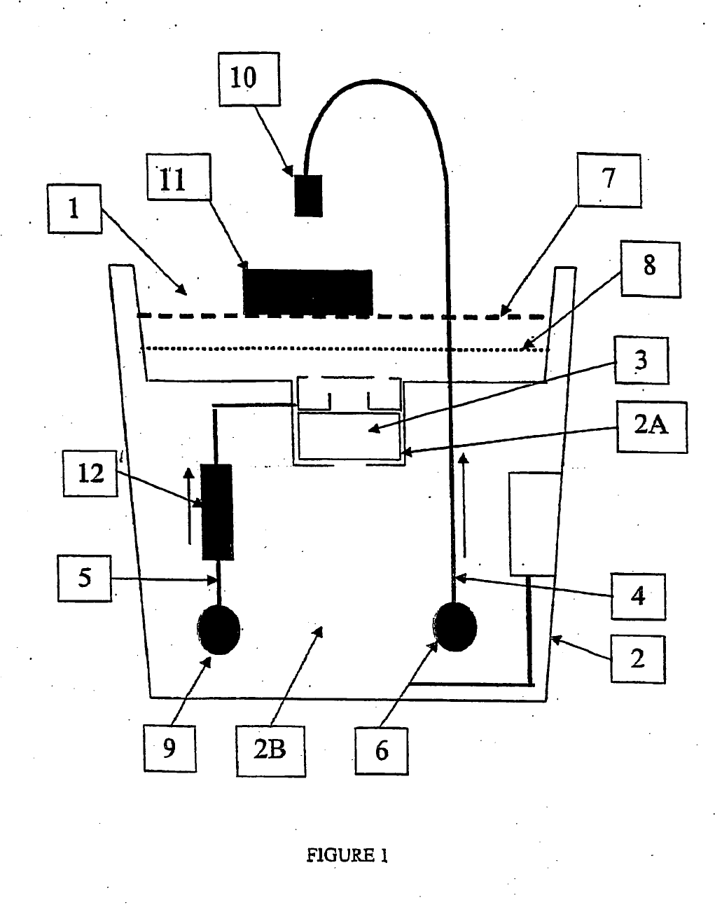

FIG. 1 is a schematic assembly view of an installation according to the invention, and

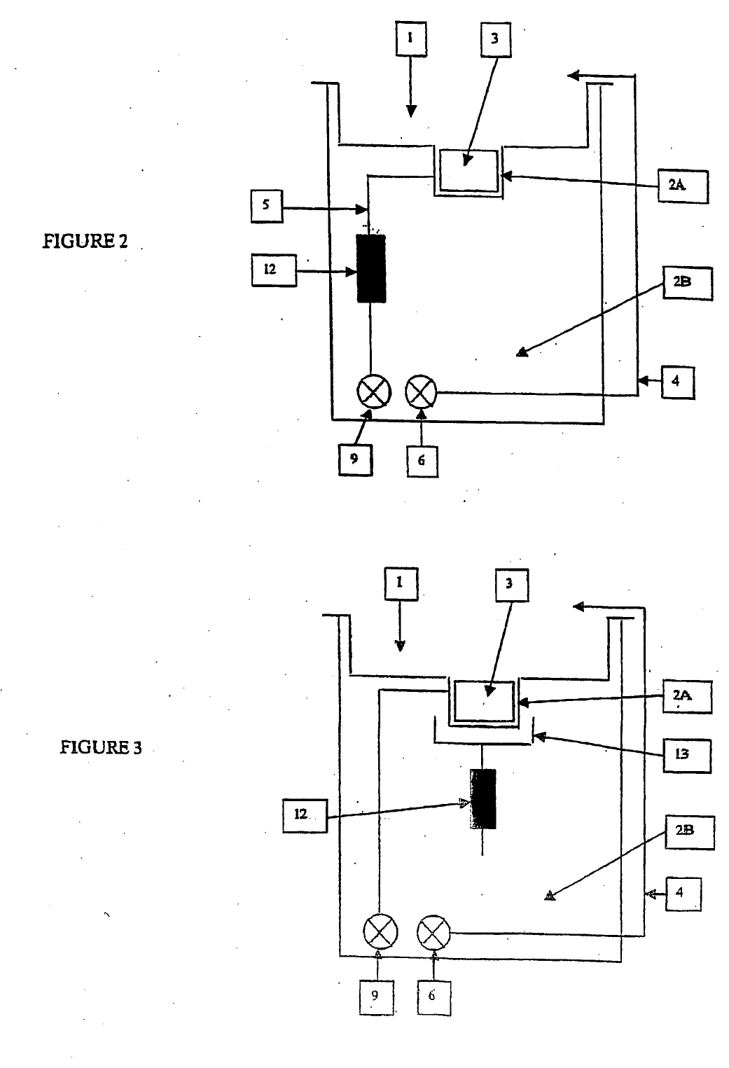

FIGS. 2 to 9 are fragmentary schematic views of various embodiments of an installation according to the invention.

As mentioned above, the installation according to the invention is more particularly adapted to permit the cleaning, in particular the degreasing or the de-oiling, of pieces soiled with organic material and bio-pollution. Such an installation is in particular adapted for the cleaning of mechanical pieces or the like. This installation comprises, in a manner known per se, a unit 1 for washing pieces in which the cleaning fluid becomes loaded with organic materials in contact with the pieces and a processing unit 2 in which living microorganisms biologically degrade the organic material contained in the fluid from washing unit 1. Obviously, by “piece” is meant any type of object.

At least a portion of the cleaning fluid is thus caused to circulate in a loop or in a closed circuit between washing unit 1 and processing unit 2. By circulating in a loop, is meant a circuit which permits the circulation of at least a portion of the fluid from washing unit 1 toward processing unit 2 on the one hand and from unit 2 toward unit 1 on the other hand to permit bringing at least a portion of the fluid from unit 2 toward unit 1. Thus, once decontaminated at least partially of organic material by the microorganisms, the cleaning fluid can be reused for the washing operation. The washing operation can take place in the form of a bath within the washing unit 1 or by projection of cleaning fluid against the surface of the pieces. It is this second case which has been selected for the embodiment shown in FIG. 1.

In a manner characteristic of the invention, this installation comprises moreover, in the fluid circulation circuit, means 12 for sterilizing at least partially microorganisms contained in at least a portion of the cleaning fluid.

Obviously, such an installation can have a large number of forms and only a few embodiments of the invention will be described hereafter. The at least partial sterilization of the cleaning fluid permits limiting or even avoiding all contact between the microorganisms contained in the cleaning fluid and the hands of the operator. Thus, the risk of contamination, injection or allergy to the operator is reduced.

These means 12 for sterilization of the cleaning fluid are preferably sterilization means by a physical mode adapted to produce heat and/or radiation and/or ultraviolet, and/or by a chemical mode for bactericidal and/or bacterio-static action on the microorganisms contained in the cleaning fluid. Thus, these sterilization means can be constituted by a UV lamp placed in the fluid circulation circuit, in particular in a conduit 4 for channeling the fluid which extends for example in the connection region between the washing unit 1 and the processing unit 2, this connection zone permitting a circulation of the cleaning fluid from the processing unit in the direction of the washing unit 1. Thus, in this case, the sterilization means 12 extend along the fluid circulation circuit, downstream of the processing unit 2. Of course, these sterilization means 12 could also be positioned in other positions of the installation as will be described hereafter. These sterilization means 12 could also be constituted by heating means for the fluid so as to give rise to destruction of the microorganisms. In this case, the heating is preferably followed by cooling of the fluid to dissipate the excess calories and to maintain the fluid at the normal use temperature between 20 and 40° C. Finally, the sterilization means can also be constituted by means for emitting radiation, in particular ultraviolet, giving rise to the destruction of microorganisms. The sterilization can also take place by a chemical mode with the help of chemical agents such as ozone. This sterilization can be total or partial. We will speak of partial or controlled sterilization when the number of microorganisms, after treatment by sterilization of the fluid, is less than a predetermined threshold but above zero. The number of microorganisms is in this case maintained below a predetermined threshold. We will speak of total or complete sterilization when there is found, after biological analysis, an absence of microorganisms in a specimen of cleaning fluid having been subjected to this step of sterilization. Moreover, this sterilization can have a bacterio-static action, which is to say simply preventing the growth of the microorganisms present in the fluid, or a bactericidal action, which is to say destroying moreover the microorganisms present in the fluid.

Although a large number of embodiments of such an installation could be given, there will be described hereafter more particularly two embodiments of the invention. In the embodiments described hereafter, the processing unit 2 traversed by the cleaning fluid from the washing unit 1 comprises at least one processing chamber, shown at 2A in the figures, filled with a filtering material 3 on which the microorganisms are immobilized and through which the cleaning fluid circulates generally in a continuous manner to ensure oxygenation of the microorganisms necessary for their development. In the illustrated example, the washing unit 1 has the form of a sink within which the pieces 11 to be treated are disposed on grating 7 itself resting on a grill 8 extending above an outlet opening for the washing unit 1. This fluid outlet positioned in the bottom of the vat or sink, is so configured as to provide, within this latter, on the one hand a cycling, on the other hand the processing chamber 2A of the treatment unit constituting a bio-reactor. Thus, this outlet orifice delimits a chamber with a generally perforated wall, integrating the filtering material 3 on which the microorganisms are immobilized. By way of example, this filtering material can be constituted by woven or non-woven cloth, geo-textile, fiber, ceramic, terra cotta, clay, plastic or glass media, open cell foam, pouzzolana, lithothamniae, metallurgical coke, silicious gravel.

The microorganisms immobilized on this support can themselves be constituted by the genus Achronobacter, Acinetobacter, Actinomyces, Alcaligenes, Bacillus, Flavobacterium, Klebsiella, Nocardia, Pseudomonas, Streptomyces, Vibrio, Xanthomyces, Aspergillus or other microorganisms that can take part in the biodegradation of hydrocarbons.

This filtering material 3 moreover contains nutrient elements for the microorganisms. These nutrient elements are preferably constituted by sources other than the carbonated sources and are preferably also insoluble, or slightly soluble, in the cleaning fluid. This constructive solution promotes the immobilization of the microorganisms on their support such that very little of the microorganism is caused to detach from the support 3 and to circulate in the fluid. The nutrient sources can be constituted by phosphorus, nitrogen, oxygen, sulfur, magnesium, potassium, calcium, iron, manganese and other oligo-elements.

In the examples shown in FIGS. 1 to 9, the processing chamber 2A of the processing unit 2, within which the aqueous cleaning fluid is treated by contact with the microorganisms, communicates with a chamber 2B for recovery and storage of the fluid at least partially purified, from the processing chamber 2A. This chamber 2B or 2B2 for the recovery of treated fluid, can be compartmented, the compartments communicating with each other by overflowing. This chamber 2B, 2B2 for recovery of treated fluid is provided on the one hand with a circuit 5 for recirculation of fluid toward the processing chamber 2A, on the other hand with means 4 for direct or indirect connection with the washing unit 1 for circulation of the fluid in the direction of the washing unit 1. These connection means 4 or circulation loop 5 are for example constituted by a conduit for circulation of a fluid within which the fluid is given to move by means of a pump.

Thus, in the examples of FIG. 1 to 9, the loop 5 for circulation of fluid between the chamber 2B or 2B2 for recovery of treated fluid and the chamber 2A for treatment of the treatment unit is constituted by a pump 9 sucking the fluid contained within the chamber 2B or 2B2 to cause it to circulate through a channel until it is introduced into chamber 2A where it can again flow through the support 3 and thus again come into contact with microorganisms adapted to treat the fluid and to decontaminate it. This fluid then returns to the chamber 2B or 2B2. In the same way, in FIGS. 1 to 7, the connection means 4, between the chamber 2B for recovery of treated fluid of the processing unit 2 and the cleaning unit 1, are constituted by a channel represented at 4 in the figures. This channel 4 is provided with a suction pump 6 for fluid contained in the chamber 2B so as to bring this fluid to within the cleaning unit 1. This fluid is thus projected by means of a brush 10 on the piece 11 to be cleaned. The cleaning fluid thus loaded with organic material is then evacuated from washing unit 1 toward processing chamber 2A of the processing unit and a new cycle can begin. The pumps 6 and 9 that are used are preferably submerged turbine pumps. This turbine is disposed within a stator adapted to create a magnetic field coacting with the magnetic axis of hydrodynamic lubrication of rotation of the turbine.

In the example shown in FIGS. 1 and 2, the sterilization means 12 are positioned in the circuit 5 for recirculation of fluid between the first and second chamber of the processing unit 2. As a modification and/or as a supplement, the sterilization means 12 could be positioned in the connection means 4 between the processing unit 2 and the washing unit 1 and/or in one of the chambers of the processing unit and/or in the connection between two chambers of the processing unit.

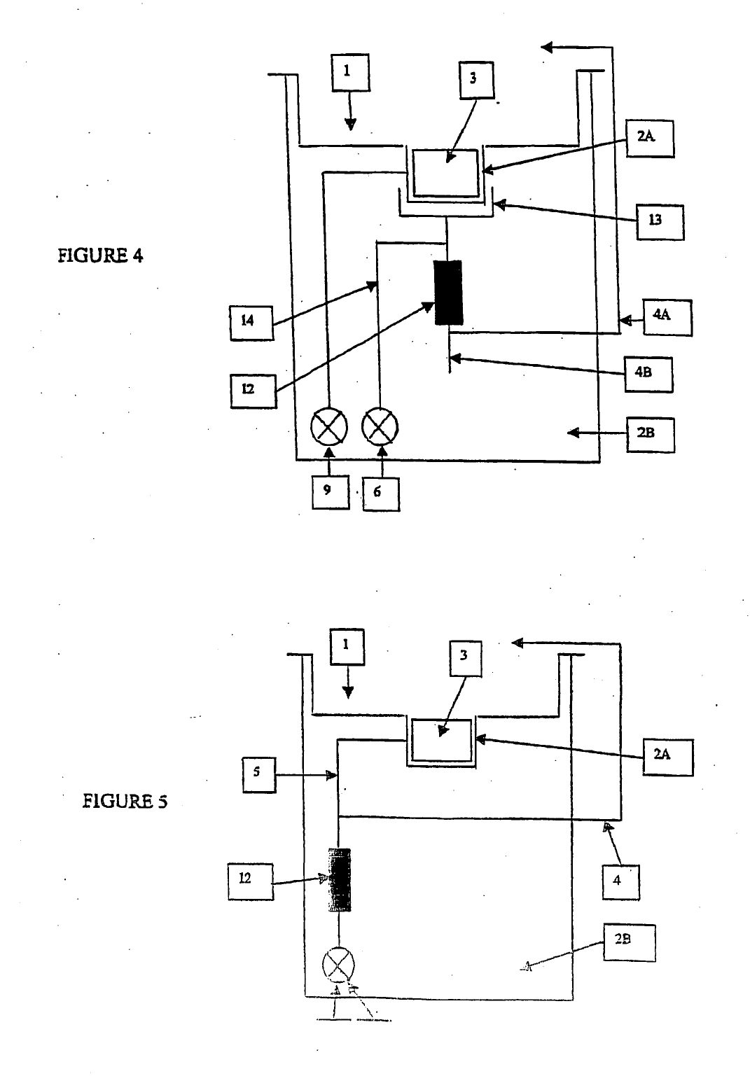

Thus, in the example shown in FIGS. 1 and 2, the fluid pumped into the chamber 2B by means of the pump 9 and recirculating through the conduit 5 to be brought to the chamber 2A of the processing unit 2, circulates through an ultraviolet radiation lamp 12 which ensures at least partial sterilization of the fluid circulating within said channel. Having regard to the quantity of fluid recirculating through this circulation loop 5, it can be considered that the fluid contained in the chamber 2B is a sterile or almost sterile fluid. In another embodiment shown in particular in FIGS. 3 and 4, the sterilization means 12 are positioned between the first and second chamber of the processing unit such that the fluid from the processing chamber 2A reaching the second chamber 2B is sterile. Thus, in the example shown in FIG. 3, there is provided a collector 13 adapted to channel the cleaning fluid from the processing chamber 2A, this channeled fluid passing through a channel provided with an ultraviolet radiation lamp 12 to sterilize the fluid adapted to flow into the second chamber 2B.

In the example shown in FIG. 4, there is seen this same collector 13 of fluid from the cleaning from the processing chamber 2A, the ultraviolet radiation lamp 12 being positioned at the outlet of this collector 13 in a conduit adapted either by its portion 4A to permit a recirculation of the fluid in the direction of the washing unit 1, or by its portion 4B to supply the chamber 2B for recovery of treated fluid. The pump 6, adapted to supply this conduit 4A that connects between chamber 2B of washing unit 1, becomes, by means of a punching 14 extending upstream of the sterilization means 12, to supply this conduit 4A. This arrangement permits guaranteeing that the fluid from the chamber 2B and directed toward the washing unit 1, has been sterilized.

As shown in FIG. 5, it is also possible to position the sterilization means immediately downstream of the pump 6 or 9 serving to suck the fluid from the recovery chamber 2B for treated fluid from the treatment unit, in the direction of treatment chamber 2A of the treatment unit. There is also provided, downstream of the circulation means, a punching permitting the connection of the container of the chamber 2B with the washing unit 1. In this case, a same pump, shown at 6 and 9 in FIG. 5, serves both for the supply of the cleaning fluid of the chamber 2A and of the washing unit 1. There can thus be arranged, downstream of the sterilization means, a circuit selector, preferably constituted by a valve actuated either manually by the operator, or automatically according to a principle of hydraulic distributor with a flap with spring return loaded to a given value, or an electro-valve controlled by manual or automatic control instructions, supplying as desired the chamber 2A or the washing unit 1.

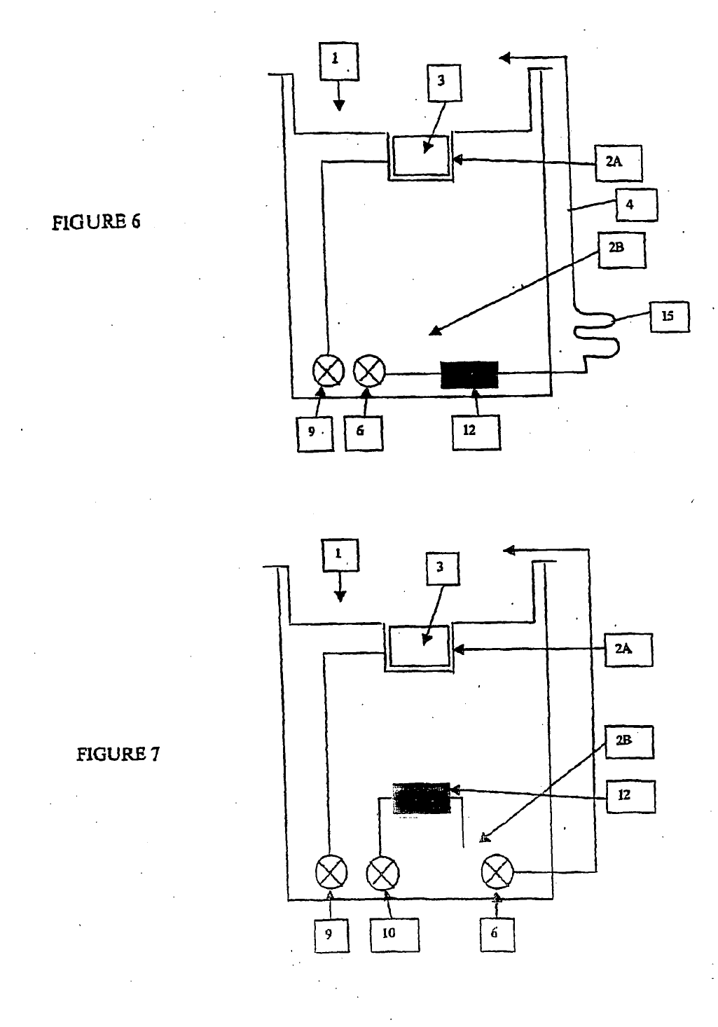

The sterilization means 12 can also be positioned in the connection channel between treatment unit 2 and washing unit 1, this connection channel 4 permitting the circulation of the cleaning fluid from the treatment unit 2 toward the washing unit 1. This example corresponds to that shown in FIG. 6, in which the sterilization means are positioned in the conduit 4 extending between the pump 6, adapted to suck the fluid into the chamber 2B, and the washing unit 1. If these sterilization means 12 are constituted by heating means, cooling means 15 are preferably associated so as to maintain the fluid at the normal use temperature between 20 and 40° C.

In another embodiment shown in FIG. 7, the sterilization means are positioned in the chamber 2B for recovery of processed fluids from the processing unit 2. Thus, the chamber 2B is maintained sterile or almost sterile by the circulation of the fluid in the sterilization means 12 by means of a pump 10 causing circulation in a loop of the fluid within said chamber.

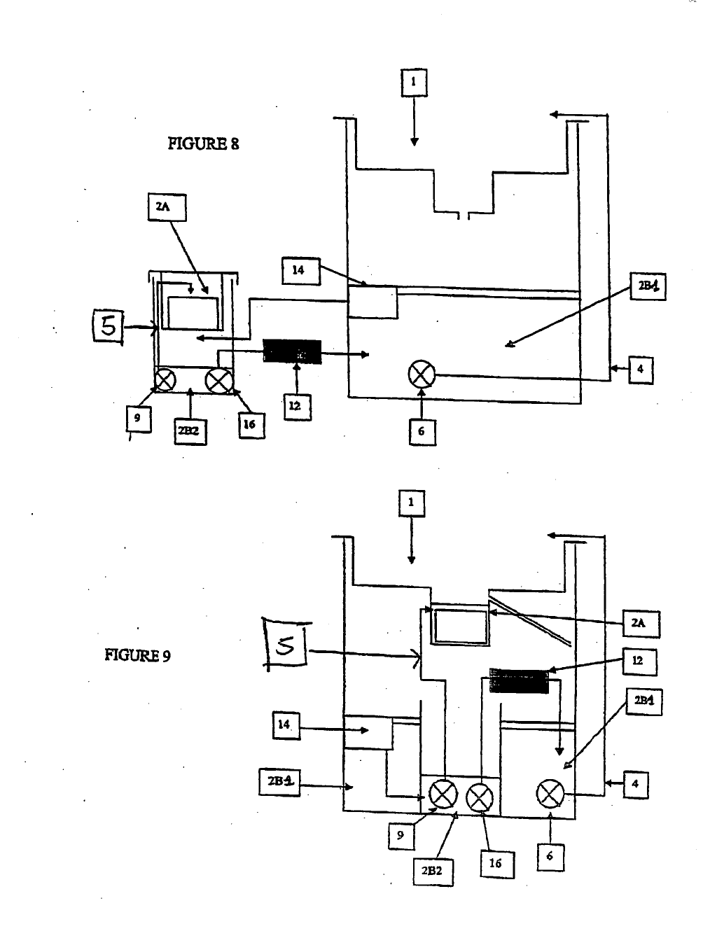

FIGS. 8 and 9 show two other embodiments of the invention in which the number of chambers of processing unit 2 has been increased. Thus, in these two embodiments, the processing chamber 2A of processing unit 2, within which the cleaning fluid is treated by contact with living microorganisms, communicates with a chamber 2B2 for recovery and storage of the fluid from the processing chamber 2A. This chamber 2B2 is provided with recirculation means for fluid toward the processing chamber 2A. This chamber 2B2 communicates with a supplemental chamber 2B1 constituting the interface of the chambers 2, 2A, 2B2 of the processing unit, with the washing unit 1. Thus, the chamber 2B2 is itself supplied with fluid from the washing unit 1 from a chamber 2B1. This interface chamber 2B1 comprises means 4 for connection with the washing unit 1 with a view toward circulation of the fluid in the direction of washing unit 1. This interface chamber 2B1 between the other chambers of the processing unit 2 and washing unit 1, supplies at least one of the other chambers of the processing unit by means for example of a de-oiling device 14. This device permits extracting the light phase of the two-phase fluid from the washing unit 1. This device comprises means for extraction of the light phase and control means as a function of said extraction means. These operational control means of the extraction means are constituted for example by, on the one hand, two floats adapted to float one to the surface of the light phase, the other to the surface of the heavy phase, and on the other hand with at least one detector whose activation permits the placing in operation or respectively the stopping of the extraction means and is subject to the relative positioning of said floats. This extraction means is for example constituted by an evacuation conduit provided at its end with a float maintaining the outflow of said conduit and in the phase to be extracted, this conduit being provided with a closure member, such as a valve controlled to open and close by said detector. The closure member is controlled to open when the distance separating the two floats of the control means, detected by said detector, is greater than a predetermined value.

Thus, in the example shown in FIG. 8, the cleaning fluid from the washing unit 1 is loaded with organic material and flows into the interface chamber 2B1 which supplies, by means of the de-oiling device 14, the chamber 2B2 provided with a recirculation circuit for fluid with the processing chamber 2A of the processing unit 2. Thus, once the fluid is treated by co-action of the chambers 2B2 and 2A, the fluid is pumped by means of a pump 16 and sent again to the interface chamber 2B1. In this connection between the chambers 2B2 and 2B1, there are provided sterilization means 12. Thus, in this case, the sterilization means are positioned on the connection between two chambers of the processing unit 2. The fluid is then pumped by means of a further pump 6 which brings it to the washing unit 1. Because of this, the chamber 2B1 never contains microorganisms, the microorganisms being lodged in the chambers 2B2 and 2A. In the example shown in FIG. 8, the chambers 2B2 and 2A moreover have been designed so as to be made in the form of a unit that is external relative to the washing unit and to the chamber 2B1 so as physically to separate the elements adapted to contain microorganisms and those normally free from microorganisms or in any case containing a quantity insufficient to cause contamination.

FIG. 9 shows another embodiment of the invention, in which the fluid from the washing unit 1 supplies the interface chamber 2B1 which itself supplies, by a de-oiling device 14, the chamber 2B2. This chamber 2B2 in communication is again provided with a circuit for recirculation of fluid with the processing chamber 2A of the processing unit 2. Once the fluid is treated, this fluid is brought to the chamber 2B1. Again, means 12 for sterilization are positioned at the connection between chambers 2B2 and 2B1. A pump 6 permits, by means of a conduit 4, to extract the fluid from this interface chamber 2B1 so as to bring it to the washing unit 1. These solutions permit avoiding in an almost complete manner a contamination with bacteria or fluid used in the washing unit 1.

As shown by all of these figures, a large number of embodiments can thus be envisaged by keeping a same objective, namely, limiting the number of living microorganisms present in the cleaning fluid adapted to supply washing unit 1. These different embodiments can be combined. It will be noted that in the examples shown in FIGS. 1 to 7, the processing chamber 2A of the processing unit 2 is positioned suspended above the other chamber 2B of the processing unit. This arrangement thus permits maintaining the support 3 from which the microorganisms are immobilized in an environment that is not immersed, so as to promote aerobic growth of the microorganisms.

1. Process for cleaning, in particular degreasing or de-oiling, pieces soiled with organic material, by means of a cleaning fluid of which at least a portion circulates in a loop between a washing unit (1) for pieces in which the cleaning fluid is loaded with organic material in contact with the pieces and a processing unit (2) in which living microorganisms biologically degrade the organic material contained in the fluid from the washing unit (1),
which process is characterized in that it consists in subjecting at least a portion of the cleaning fluid circulating in said installation to at least partial sterilization so as to limit or even to eliminate the presence of living microorganisms in the cleaning fluid used in the washing unit (1).

2. Installation for cleaning, in particular degreasing or de-oiling, pieces soiled with organic material by means of a cleaning fluid of which at least a portion circulates in a loop between a washing unit (1) for pieces in which the cleaning fluid becomes loaded with organic material in contact with the pieces, and a processing unit (2) in which the living microorganisms biologically degrade the organic material contained in the fluid from washing unit (1),
characterized in that the installation moreover comprises, in the fluid circulation circuit, means (12) for at least partially sterilizing microorganisms contained in at least one portion of the cleaning fluid.

3. Installation according to claim 2,
characterized in that the means (12) for sterilization of the cleaning fluid are sterilization means by physical mode adapted to produce heat and/or radiation and/or ultraviolet and/or by chemical mode so as to have a bactericidal and/or bacterio-static action on the microorganisms contained in said cleaning fluid.

4. Installation according to claim 2,
characterized in that the processing unit (2) traversed by the cleaning fluid from the washing unit (1) comprises at least one processing chamber (2A) filled with a filtering material (3) on which the living microorganisms are immobilized and through which the cleaning fluid circulates.

5. Installation according to claim 4,
characterized in that the filtering material (3) moreover contains nutrient elements for the microorganisms, these nutrient elements, preferably constituted by sources other than the carbonated sources, being preferably insoluble or slightly soluble in the cleaning fluid.

6. Installation according to claim 4,
characterized in that the processing chamber (2A) of the processing unit (2) within which the cleaning fluid is treated by contact with living microorganisms, communicates with a chamber (2B) for recovery and storage of the fluid from the processing chamber (2A), this chamber (2B) being provided on the one hand with a circuit (5) for recirculation of fluid toward the processing chamber (2A), on the other hand by means (4) for connection with the washing unit (1) for circulation of the fluid in the direction of the washing unit (1).

7. Installation according to claim 4,
characterized in that the processing chamber (2A) of the processing unit (2) within which the cleaning fluid is treated by contact with living microorganisms, communicates with a chamber (2B2) for recovery and storage of the fluid from the first treatment chamber (2A), this chamber (2B2), provided with a circuit (5) for recirculation of fluid toward the treatment chamber (2A), being itself in communication with a supplemental chamber (2B1) constituting an interface of the other chambers of the processing unit with the washing unit, this interface chamber (2B1) comprising means (4) for connection with the washing unit (1) to provide circulation of fluid in the direction of the washing unit (1).

8. Installation according to claim 7,
characterized in that the interface chamber (2B1) between the other chambers of the processing unit (2) and the washing unit (1) supplies with fluid the other chambers of the processing unit by means of a de-oiling device (14).

9. Installation according to claim 6,
characterized in that the sterilizing means (12) are positioned in the connection between two chambers of the processing unit, in particular in the circuit (5) for recirculation of fluid between the processing chamber (2A) and the chamber (2B, 2B2) for recovery and storage of the fluid from the processing chamber (2A) and/or in the connection means (4) between the processing unit (2) and the washing unit (1) and/or in one of the chambers of the processing unit (2).

10. Installation according to claim 9,
characterized in that the sterilization means (12) are positioned between the processing chamber (2A) and the chamber (2B, 2B2) for recovering storage of fluid processed in the processing unit (2) such that the fluid from the processing chamber (2) reaching the other chamber (2B, 2B2) is sterile.

11. Installation according to claim 8,
characterized in that the processing chamber (2A) of the processing unit (2) is positioned suspended above the chamber (2B, 2B2) for storage of fluid processed in the processing unit (2).

12. Installation according to claim 4,
characterized in that the washing unit (1) is provided with an outlet for the evacuation of fluid in the interior of which is provided the processing chamber (2A) of the processing unit (2).

13. Installation according to claim 1,
characterized in that the processing unit (2) is provided with connection means (4) with the washing unit (1) for circulation of the fluid in the direction of the washing unit (1), the sterilization means (12) being positioned in the connection channel between said units (2, 1).

14. Installation according to claim 3,
characterized in that the processing unit (2) traversed by the cleaning fluid from the washing unit (1) comprises at least one processing chamber (2A) filled with a filtering material (3) on which the living microorganisms are immobilized and through which the cleaning fluid circulates.

15. Installation according to claim 7,
characterized in that the sterilizing means (12) are positioned in the connection between two chambers of the processing unit, in particular in the circuit (5) for recirculation of fluid between the processing chamber (2A) and the chamber (2B, 2B2) for recovery and storage of the fluid from the processing chamber (2A) and/or in the connection means (4) between the processing unit (2) and the washing unit (1) and/or in one of the chambers of the processing unit (2).

16. Installation according to claim 9,
characterized in that the processing chamber (2A) of the processing unit (2) is positioned suspended above the chamber (2B, 2B2) for storage of fluid processed in the processing unit (2).

17. Installation according to claim 10,
characterized in that the processing chamber (2A) of the processing unit (2) is positioned suspended above the chamber (2B, 2B2) for storage of fluid processed in the processing unit (2).

18. Installation according to claim 5,
characterized in that the washing unit (1) is provided with an outlet for the evacuation of fluid in the interior of which is provided the processing chamber (2A) of the processing unit (2).

19. Installation according to claim 6,
characterized in that the washing unit (1) is provided with an outlet for the evacuation of fluid in the interior of which is provided the processing chamber (2A) of the processing unit (2).

20. Installation according to claim 7,
characterized in that the washing unit (1) is provided with an outlet for the evacuation of fluid in the interior of which is provided the processing chamber (2A) of the processing unit (2).
